(19) United States
(12) Patent Application Publication
(10) Pub. No.: US 2021/0000409 A1
(43) Pub. Date: Jan. 7, 2021

(54) DEVICES FOR DETECTING AND/OR IDENTIFYING MICROBIOLOGICAL INFECTIONS, FOR NON-IMPLANTABLE MEDICAL DEVICES

(71) Applicant: BIOMÉRIEUX, Marcy L'Etoile (FR)

(72) Inventors: Fanny BERRAT, Tassin La Demi Lune (FR); Aline DARDY, Lyon (FR); Christine ROZAND, Saint Genis les Ollieres (FR)

(73) Assignee: BIOMÉRIEUX, Marcy L'Etoile (FR)

(21) Appl. No.: 16/768,790

(22) PCT Filed: Nov. 29, 2018
(86) PCT No.: PCT/FR2018/053034

§ 371 Date: Jun. 1, 2020

(30) Foreign Application Priority Data
Nov. 30, 2017 (FR) 1761403

(51) Int. Cl. A61B 5/00; A61L 26/00; A61F 13/02; A61F 13/00; A61B 5/145

(52) U.S. Cl. CPC A61B 5/445; A61L 26/008; A61L 26/0014; A61B 2562/02; A61F 13/00063; A61B 5/14546; A61L 2300/406; A61F 13/0206

(57) ABSTRACT

A biosensor intended to be integrated in the actual structure of a dressing, a compress or any other similar absorbent article. This biosensor makes it possible to monitor the onset of a microbiological infection of a wound and/or of a skin lesion. The biosensor includes a piece of absorbent hydrophilic material on which, on the surface and/or within the thickness thereof, a composition of agglomerated powders is immobilized, the composition including ethylene-vinyl acetate (EVA) particles having a surface partially coated at least with a salt of orthophosphoric acid and a visual indicator of microbiological growth.

The present invention is in the field of microbiological diagnostics and finds application in the field of nonimplantable medical devices used for covering wounds and skin lesions to protect them from contaminants and external infections, such as dressings, compresses and other similar absorbent articles. The present invention relates more precisely to means for diagnosis of microbiological infections, intended to be incorporated in said nonimplantable medical devices, in order to detect the onset of a possible infection.

Wounds, regardless of their severity, are subject to risks of infection by pathogenic microorganisms such as bacteria, viruses, fungi, and protozoa. Besides interrupting the physiological process of wound-healing, these infections may lead to serious complications, such as gas gangrene, tetanus, and may give rise to long-term disabilities (chronic infection of the wound or bone, etc.), or even death following sepsis.

In the case of hospitalization, more than 90% of the postoperative infections seen occur within 30 days following a surgical procedure. Patients must therefore be monitored for at least 30 days after their operation. Clinically, this monitoring generally consists of visual examination of the healing of wounds and observation of any purulent discharge, which depending on its color, consistency and odor may be a sign of infection. When an infection is suspected, a specimen is obtained and is then sent to the analytical laboratory for additional investigations: microscopic analyses, microbiological cultures and/or antibiograms etc. In practice, the practitioner will not receive the analysis results until the next day, at the earliest.

For better monitoring of patients, health care professionals (doctors, practitioners, nurses, etc.) have expressed the desire to have tests and diagnostic tools that enable them to establish for themselves the existence of an infection, and optionally envisage the treatment to prescribe, actually during the consultation.

In this context, it was envisaged to integrate means for detecting and/or diagnosing microbiological infections in the structure of compresses, dressings and other nonimplantable medical devices used for covering and protecting wounds.

In a first approach, on the basis of recent technological advances in the field of microelectrodes, electrochemical sensors have been developed for carrying out in situ electrochemical analyses of the chemical composition of the exudates from wounds, with a view to detecting the presence of biomarkers associated with microbiological infections.

In this context, we may notably mention the works of Hajnsek et al., 2015 (SENSOR AND ACTUATORS B: CHEMICAL; 265-274: “An electrochemical sensor for fast detection of wound infection based we myeloperoxidase activity”) and Sismaet et al., 2016 (WOUND REPAIR AND REGENERATION; (24)366-372: “Electrochemical detection of Pseudomonas in wound exudate samples from patients with chronic wounds”). In the first example, the sensor allows electrochemical detection of myeloperoxidase activity, which is expressed by many pathogenic bacteria. In the second example, the sensor allows electrochemical detection of pyocyanine, a molecule produced by Pseudomonas aeruginosa.

These electrochemical sensors are currently at an early experimental stage of their development. They incorporate miniaturized electrode technology, the present cost of which is incompatible with the dressings and compresses market. Moreover, these electrochemical sensors are not autonomous. To function, they must be connected to a whole range of external equipment for electric power supply, for collecting the data transmitted by the sensors, and for processing the data before being able to deliver an analysis result to the practitioner; the latter will have to have so much equipment at his disposal.

In a second approach, more in keeping with the technological and economic constraints of the dressings and compresses market, the concept of a “smart bandage” has been imagined. Placed on the wound, it would generate a visible signal (appearance of a color and/or of fluorescence) if there is a microbiological infection.

In this context, Brocklesby et al., 2013 (Medical hypotheses; (80)237-240: “Smart bandages—A colorful approach to early stage infection detection & control the wound care”) envisages the production of a bandage allowing detection of β-lactamase-producing bacteria. For this purpose, it is suggested to use a chromogenic “chimeric” cephalosporin, assembled from a cephalosporin residue and a chromophore. This chromogenic “chimeric” cephalosporin would be fixed by chemical coupling to the fibers of the absorbent textile of the bandage. Its hydrolysis by cephalosporinases would cause coloration of the bandage. To date, no device or dressing functioning on this principle of detection has been produced.

More recently, researchers at the University of Bath in the United Kingdom announced the development of a prototype of a smart bandage emitting fluorescence in response to a bacterial infection (Thet et al., 2016—ACS Applied Materials & Interfaces; 8(24):14909-19: “Prototype development of the intelligent hydrogel wound dressing and its efficacy in the detection of model pathogenic wound biofilm”).

This dressing is formed from a piece of absorbent cloth, the face of which that is intended to be applied directly in contact with the patient's wound is covered with a 2% agarose hydrogel. Vesicles loaded with 5,6-carboflurescein are distributed in the bulk of this hydrogel. The wall of these vesicles consists of an assemblage of phospholipids, cholesterol and polyacetylene polymers. By its composition, it mimics the phospholipid membrane of eukaryotic cells and is perforated by the cytotoxins that the pathogenic bacteria release during an infection. For its part, 5,6-carboflurescein is a compound that has two interesting features, ingeniously exploited in this device. The first is that it self-assembles into dimers when its concentration is high enough, and then dissociates as its concentration decreases. The second feature is that it emits fluorescence when it is in the monomeric state; in the dimeric state, in contrast, its fluorescence is blocked.

In the case of bacterial infection, the wall of the vesicles is broken by the cytotoxins that are released, and the 5,6-carboflurescein is released from the vesicles and is gradually diluted in the hydrogel. When exposed to UV light, the dressing then emits fluorescence.

At present, this device is at the prototype stage and many tests are required to ensure safety of the agarose hydrogel, and of the vesicles and the 5,6-carboflurescein, which will be in direct contact with the patient's wound.

There are some major drawbacks with such a device. A first drawback is connected with the nonspecificity of detection. Such a device will only be able to alert the practitioner to the onset of an infection. Additional tests will have to be carried out if he wants to know the nature of the pathogen(s) involved and prescribe a specific treatment. A second drawback arises from the stability over time of the agarose hydrogel and is based more on considerations relating to industrial and commercial exploitation of such a device. Owing to the presence of this hydrogel, the commercial success of such a device will suffer from a short shelf life, and considerable constraints on packaging and storage.

The present invention aims to overcome the aforementioned drawbacks. Thus, its general aim is to propose a technical solution for making so-called smart dressings, notably in terms of simplicity of application and specificity of detection.

One aim of the present invention is to propose means for diagnosis of microbiological infections that can be incorporated mainly in structures such as dressings, compresses and other absorbent articles intended to cover wounds and skin lesions, and would be able to signal a state of bacterial infection by a color change and/or by the emission of fluorescence. Another aim of the present invention is to allow detection and specific identification of the pathogen or pathogens in question, owing to the appearance of profiles of coloration and/or of emission of fluorescence that are specific and characterizable.

It is the ambition of the present invention to propose means for in situ diagnosis of microbiological infections, which may be transferable/applicable to the industry of dressings, compresses, etc., but also to related technical fields (in particular, absorbent articles of the disposable diaper and sanitary pad type, used for medical purposes), and compatible with the constraints of industrial and commercial exploitation typical of this sector.

Finally, the present invention aims to propose technology that can be exploited both for human medicine and for veterinary medicine.

In this context, the present invention proposes means for in situ diagnosis of microbiological infections in the form of a biosensor comprising a piece of absorbent hydrophilic material on which, on the surface and/or within the thickness thereof, a composition of agglomerated powders is immobilized, said composition comprising ethylene-vinyl acetate (EVA) particles, the surfaces of which are partially coated with at least one salt of orthophosphoric acid and a visual indicator of microbiological growth. In the presence of microorganisms, especially bacteria and yeasts, said visual indicator of microbiological growth is able to produce a signal visible to the naked eye when the biosensor is exposed to white light and/or to UV radiation.

A biosensor according to the invention is intended to be incorporated into the actual structure of a dressing, a compress or any other similar absorbent article for covering wounds and skin lesions. Positioned near a wound or a lesion, the biosensor is designed to be able to capture and collect the exudate from wounds, and form a substrate that promotes the growth and development of microorganisms. Detection and/or identification of any microorganisms present are based on visual indicators (chromogens and/or fluorogens) of microbiological growth, sensitive to the metabolic activity of the microorganisms in question.

To design a biosensor of this type, the inventors employed the techniques of in vitro microbiology for detecting and identifying microorganisms such as bacteria, yeasts, molds and amoebae, and took inspiration more particularly from work conducted since the 1990s on chromogenic and fluorogenic culture media (Perry et al., 2007—Journal of Applied Microbiology; 103:2046-55: “The application of chromogenic media in clinical microbiology.”).

From the composition of these culture media, the inventors essentially adopted and applied the teachings concerning the visual indicators of microbiological growth, use of which allows visual demonstration of the microorganisms via their metabolic activity. Numerous visual indicators of microbiological growth, usable for making a biosensor according to the invention, are reported in the literature and are available commercially. They may be, for example, colored indicators of pH, sensitive to the change in pH induced by hydrolysis of sugars or of other substrates metabolizable by the targeted microorganisms. More preferably, they are chromogenic and/or fluorogenic substrates that are able to release a chromophore and/or fluorophore compound under the action of a particular enzyme activity (such as for example β-glucuronidase, β-galactosidase, β-glucosidase, α-glucuronidase, α-galactosidase, α-glucosidase, esterase, nitroreductase, phosphatases, aminopeptidase activities etc.).

To eliminate any risk of superinfection of the wound that might be attributable to the biosensor, the inventors took care, in designing the latter, to exclude any ingredient having nutrient potential with respect to microorganisms. Advantageously, the composition of agglomerated powders immobilized in the biosensor according to the invention does not have any notable/measurable nutrient quality for the growth and development of microorganisms. In particular, this composition of agglomerated powders is free from any physiologically effective amount of carbohydrates, lipids, amino acids and growth factors, supplied in the form of chemically well-defined compositions or in the form of complex compositions such as mixtures of peptones, yeast extracts, serums etc.

Thus, with a biosensor according to the invention, in case of microbiological infection of wounds, the microorganisms can only draw the nutrients necessary for their development from the exudate from the wound, the only nutrient source available in this particular environment.

For incorporating and immobilizing, in the absorbent material, an amount of visual indicator of microbiological growth that is operative for detecting and/or identifying the target microorganisms, it was necessary to find an adhesive that meets the following three main conditions:
it should be capable of effectively immobilizing the visual indicators of microbiological growth on the absorbent hydrophilic material, without impairing their functioning or impeding their hydrolysis in the case of infection by targeted microorganisms,
it should be sufficiently effective so that the amount to be used is not detrimental to the absorbency of the absorbent hydrophilic material,
it should be physiologically neutral with respect to the microorganisms, i.e. it should not have notable activating or inhibitory effects on the growth and development of the microorganisms.

None of the adhesive materials tested by the inventors could fully meet these three conditions. Nevertheless, among the many materials tested, EVA was of considerable interest to the inventors.

A synthetic resin that has been well-known since the 1950s for its thermoplastic and thermosetting properties, EVA is prepared by copolymerization of ethylene with vinyl acetate (the content of vinyl acetate by weight generally varies between 10 and 40%). It is used in a great variety of forms in many sectors of industry (for example, plastic film, heat-sealing adhesive for packaging, glue in bookbinding, a surface layer of glazed paper for magazines, a plasticizer in paint and varnish compositions, a component of a thermo-molded plastic material, a component of a composition for encapsulation of active pharmaceutical ingredients, etc.). In the context of the development of the biosensors according to the invention, the inventors were able to find very simple manipulation and use of EVA. Heating to temperatures of the order of 50-60° C. is able to soften these particles and make their surface sticky. When heated to these temperatures, the EVA particles have good surface adherence for a large number of compounds, in particular for the particles of visual indicators of microbiological growth, which they can immobilize and expose on their surface. Unfortunately, the inventors also found bacteriostatic activity of EVA, making it unsuitable for application in the field of microbiology and even more so when the end use is a cell culture for purposes of detection and/or identification. However, the inventors persevered and succeeded in overcoming this obstacle, and demonstrated that the addition of a salt of orthophosphoric acid makes it possible to inhibit this undesirable bacteriostatic effect.

Advantageously, according to the invention, the salt(s) of orthophosphoric acid used for inhibiting the bacteriostatic effect of EVA is (are) selected from potassium dihydrogen phosphate (KH₂PO₄) and sodium dihydrogen phosphate (NaH₂PO₄).

According to a first embodiment, potassium dihydrogen phosphate is used for inhibiting the bacteriostatic effect of EVA. The KH₂PO₄/EVA weight ratio is between 1:25 and 1:8, preferably between 1:16 and 1:12.

According to a second embodiment, sodium dihydrogen phosphate is used for inhibiting the bacteriostatic effect of EVA. The NaH₂PO₄/EVA weight ratio is between 1:15 and 1:3, preferably between 1:10 and 1:5.

According to a third embodiment, KH₂PO₄ and NH₂PO₄ are used concomitantly for inhibiting the bacteriostatic effect of EVA.

Advantageously, according to the invention, the EVA has a vinyl acetate content by weight of 10 to 40%, preferably of the order of 20 to 35%.

Advantageously, according to the invention, the surface of the EVA particles is also partially coated with a gelling agent which, after absorption of the exudate, gives a gel. Using a gelling agent in this particular context offers several advantages. Firstly, it improves the absorption performance of the piece of absorbent hydrophilic material that forms part of the biosensor. Moreover, it makes it possible to retain and immobilize the microorganisms that are captured.

Many gelling agents may be used for this purpose, notably agar, agarose, guar gum and xanthan gum. Xanthan gum is preferred.

According to a particular embodiment of a biosensor according to the invention, the surface of the EVA particles may also be partially coated with a selective agent. This selective agent is selected for blocking or slowing the development of the subsidiary flora, rather than that of a target microorganism or of a target group of microorganisms. Essentially they are compounds with antibiotic and/or antifungal effects, having a certain specificity of toxicity (lower toxicity for the target microorganisms than for the subsidiary flora).

Regarding the piece of absorbent hydrophilic material constituting a biosensor according to the present invention, a great many materials may be used. Advantageously, a fibrous material is used, made up of nonwoven fibers, forming an assembly having integrity and mechanical cohesion. Said fibers may be natural cellulose fibers (such as cotton) or synthetic cellulose fibers (such as rayon), modified cellulose fibers (for example, carboxymethylcellulose, nitrocellulose), chemical polymer fibers (such as polyacrylate salts, acrylate/acrylamide copolymers).

Advantageously, said piece of absorbent material has a weight per unit area between 50 g·m⁻² and 200 g·m⁻², and a thickness advantageously between 0.5 mm and 4 mm.

According to a preferred embodiment, this piece of absorbent hydrophilic material is made from a nonwoven textile, for example based on cellulose fibers. According to this preferred embodiment, the composition of agglomerated powders is advantageously fixed to the fibers of the material, essentially within its thickness.

The incorporation of a dry composition within the thickness of a fibrous substrate may be carried out in various ways, notably by so-called dry impregnation techniques. For this purpose, a dry pulverulent composition is dusted on the surface of a porous substrate, and then the particles of said composition are caused to vibrate, under the action of ultrasonic waves (cf. for example, FR 2 866 578) or else under the action of an alternating electric field (cf. for example, WO 2015/044605, WO 2010/001043 or WO 99/22920). These particles penetrate and then gradually sink into the cavities of the porous body.

For making a microbiological culture device according to the invention, the techniques of dry impregnation using an alternating electric field proved particularly suitable for allowing a powder to be incorporated within the thickness of a hydrophilic absorbent material having a fibrous structure. Accordingly, the technical teachings provided by WO 2015/044605, WO 2010/001043 and WO 99/22920 form an integral part of the present description.

Advantageously, the amount of composition of agglomerated powders incorporated in the bioreactor according to the invention is equivalent to a concentration between 10 mg·cm⁻³ and 100 mg·cm⁻³, preferably between 30 mg·cm⁻³ and 50 g·cm⁻³.

Once dry impregnation has been carried out, a thermal treatment makes the EVA sticky and fixes the particles of agglomerated powders to the fibers of the absorbent substrate. This thermal treatment advantageously consists of a calendering operation. Owing to the pressure and the heating temperature applied, calendering makes it possible to fix and permanently retain, within the thickness of the piece of absorbent hydrophilic material, the EVA particles with the visual indicator(s) of microbiological growth exposed on their surface. Calendering also improves the flatness of the surface of the piece of absorbent hydrophilic material and increases its capacity for capillarity and absorption.

Calendering is advantageously carried out at a temperature between 50° C. and 80° C.

The present invention also extends to a composition of agglomerated powders. Said composition of agglomerated powders comprises EVA particles having a surface partially coated at least with a salt of orthophosphoric acid and a visual indicator of microbiological growth.

Advantageously, according to the invention, the salt(s) of orthophosphoric acid is (are) selected from potassium dihydrogen phosphate (KH₂PO₄) and sodium dihydrogen phosphate (NaH₂PO₄).

According to a first embodiment, potassium dihydrogen phosphate is used in a KH₂PO₄/EVA weight ratio between 1:25 and 1:8, preferably between 1:16 and 1:12.

According to a second embodiment, sodium dihydrogen phosphate is used in an NaH₂PO₄/EVA weight ratio between 1:15 and 1:3, preferably between 1:10 and 1:5.

According to a third embodiment, KH₂PO₄ and NaH₂PO₄ are present concomitantly on the surface of the EVA particles.

A composition of agglomerated powders according to the invention is quite particularly useful for the biosensors according to the invention; it greatly facilitates their manufacture.

Advantageously, according to the invention, the pulverulent mixture made up of the salt(s) of orthophosphoric acid and the visual indicator(s) of microbiological growth and whose particles are intended to cover the surface of the EVA particles, also comprises a gelling agent. Said gelling agent may be agar, agarose, guar gum or xanthan gum. According to this embodiment, the surface of the EVA particles is also partially coated with said gelling agent. Xanthan gum is preferred.

Advantageously, according to the invention, this pulverulent mixture may also comprise an agent that stimulates the metabolism of bacteria, for example manganese chloride (MnCl₂), and/or an agent selective for antibiotic and/or antifungal effects. According to this embodiment, the surface of the EVA particles is also partially coated with an agent that stimulates the metabolism of bacteria. MnCl₂ is preferred.

Advantageously, according to the invention, the EVA particles of the composition of agglomerated powders according to the invention has a granulometry between 40 and 150 μm. The particles of salt(s) of orthophosphoric acid, the particles of visual indicator(s) of microbiological growth, optionally particles of gelling agent, particles of agent stimulating the metabolism of bacteria and particles of selective agent, which cover the surface of these EVA particles, have a granulometry advantageously between 5 and 50 μm.

The invention also relates to a biosensor, a composition of agglomerated powders as well as any nonimplantable medical device (such as dressings, compresses, etc., but also disposable diapers and sanitary pads used for purposes of human or animal medical monitoring) incorporating in its structure said biosensor and/or said dry pulverulent composition, characterized by some or all of the technical features disclosed above and hereunder.

Other aims, features and advantages of the invention will become clear from the description given hereunder and the examples presented below, which relate to the appended figures, where:

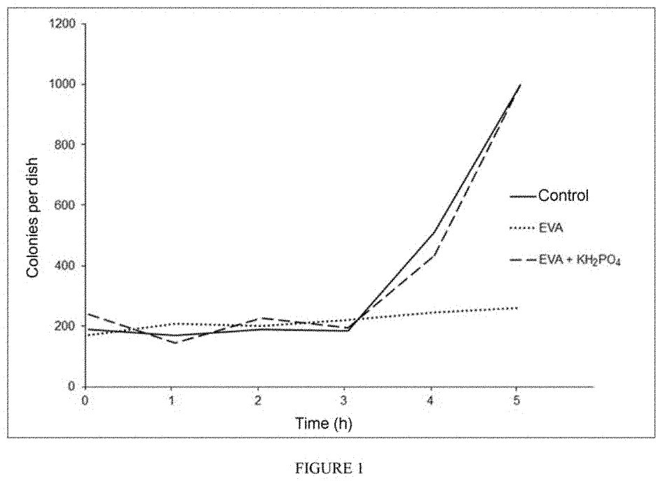

FIG. 1 is a graphical representation of the bacteriostatic effect of EVA on the development of bacteria, and inhibition of this bacteriostatic effect by the salts of orthophosphoric acid;

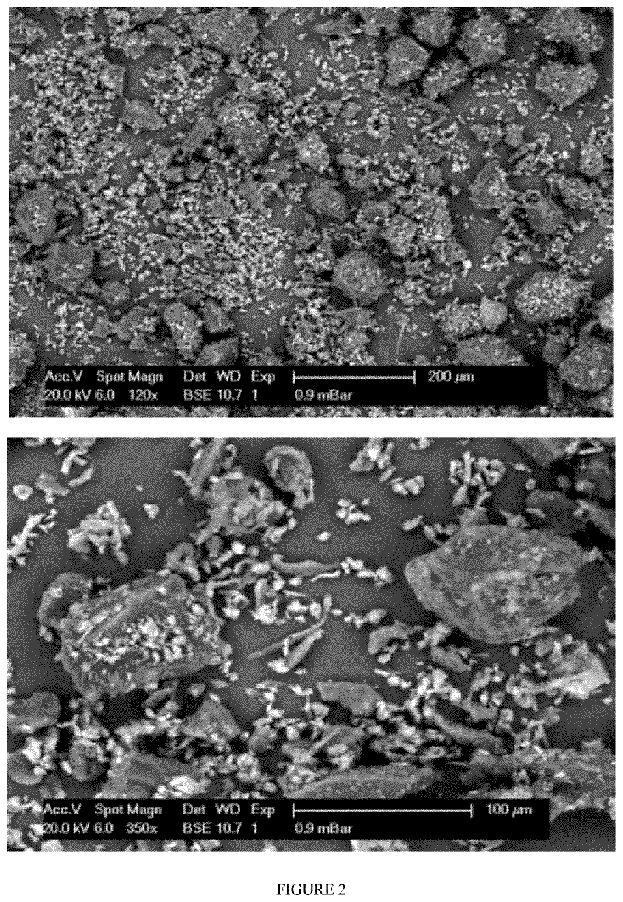

FIG. 2 corresponds to two photographs, taken by scanning electron microscopy, showing a pulverulent composition of dissociated particles;

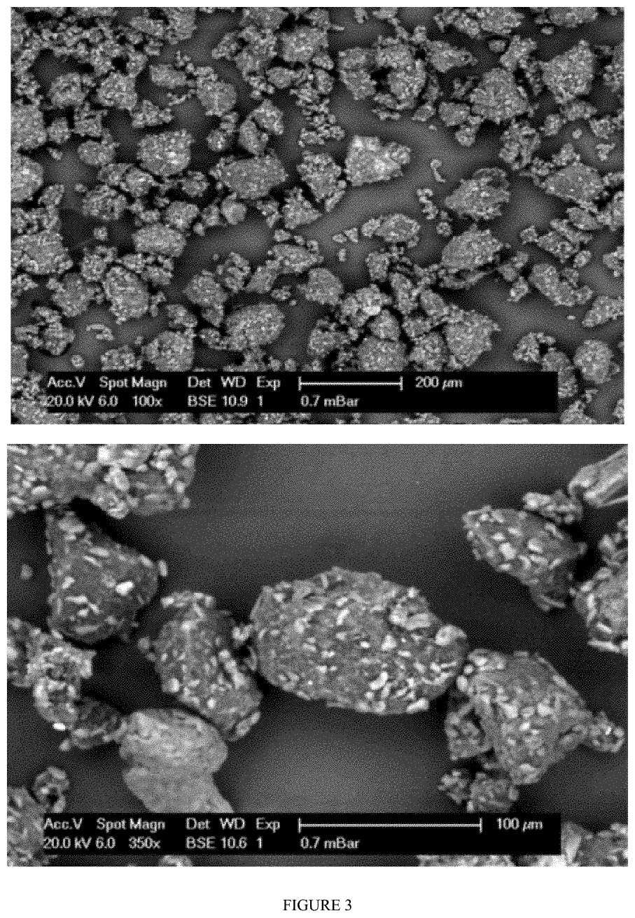

FIG. 3 corresponds to two photographs, taken by scanning electron microscopy, showing a composition of agglomerated powders according to the invention.

### EXAMPLES

### Example 1: Demonstration of the Bacteriostatic Effect of EVA and Inhibition of this Effect by Adding Salts of Orthophosphoric Acid (KH₂PO₄, NaH₂PO₄)

A. Demonstration Performed on Agar Culture Media

The effects of EVA and of the salts of orthophosphoric acid (more particularly, KH₂PO₄ and/or NaH₂PO₄) on bacterial growth and development were evaluated, firstly, on agar culture media, namely:
a tryptone soy agar (TSA), a universal medium known to be suitable for culture and enrichment of most bacteria, whether they are aerobic or anaerobic;
SAID® agar (bioMérieux, France), a culture medium marketed for the specific detection of S. aureus,

For this purpose, strains of S. aureus obtained from the applicant's collection were used. Before inoculating them in the two agars mentioned above, the bacteria (100 mL of a suspension of 10³ CFU.mL⁻¹) were resuspended beforehand in human serum prepared and supplied by Etablissement Francais du Sang (EFS, the French National Blood Service), with or without supplementing with EVA and/or with salt of orthophosphoric acid as follows:
s01: serum (EFS, reference 4566200/4566201)
s02: serum s01 with addition of EVA (to a final concentration of 53 g·L⁻¹)
s03: serum s01 with addition of EVA and NaH₂PO₄ (to final concentrations of 53 g·L⁻¹ and 4.5 g·L⁻¹ respectively)
s04: serum s01 with addition of EVA and NaH₂PO₄ (to final concentrations of 53 g·L⁻¹ et 9.0 g·L⁻¹ respectively)
s05: serum s01 with addition of NaH₂PO₄ (to a final concentration of 4.5 g·L⁻¹)
s06: serum s01 with addition of NaH₂PO₄ (to a final concentration of 9.0 g·L⁻¹)
s07: serum s01 with addition of EVA and KH₂PO₄ (to final concentrations of 53 g·L⁻¹ and 10.6 g·L⁻¹ respectively)
s08: serum s01 with addition of EVA and KH₂PO₄ (to final concentrations of 53 g·L⁻¹ and 1.5 g·L⁻¹ respectively)
s09: serum s01 with addition of EVA and KH₂PO₄ (to final concentrations of 53 g·L⁻¹ et 3.0 g·L⁻¹ respectively)
s10: serum s01 with addition of KH₂PO₄ (to a final concentration of 1.5 g·L⁻¹)
s11: serum s01 with addition of EVA, NaH₂PO₄ and KH₂PO₄ (to final concentrations of 53 g·L⁻¹, 4.5 g·L⁻¹ and 1.5 g·L⁻¹ respectively)
s12: serum s01 with addition of EVA, NaH₂PO₄ and KH₂PO₄ (to final concentrations of 53 g·L⁻¹, 9.0 g·L⁻¹ and 3.0 g·L⁻¹ respectively)
s13: serum s01 with addition of NaH₂PO₄ and KH₂PO₄ (to final concentrations of 4.5 g·L⁻¹ and 1.5 g·L⁻¹ respectively)
s14: serum s01 with addition of NaH₂PO₄ and KH₂PO₄ (to final concentrations of 9.0 g·L⁻¹ and 3.0 g·L⁻¹ respectively).

The EVA used in these tests is marketed by the company DAKOTA, Belgium, in the form of a powder with the reference UNEX EVA (EVA T1). Its vinyl acetate content by weight is 28%.

The results obtained are presented in Table 1 below.

TABLE 1

| Sera | TSA | SAID |
|---|---|---|
| s01 | bacterial lawn | bacterial lawn |
| s02 | 1 colony | Nothing |
| s03 | >300 colonies | >300 colonies |
| s04 | 300 colonies | 300 colonies |
| s05 | bacterial lawn | bacterial lawn |
| s06 | bacterial lawn | bacterial lawn |
| s07 | nothing | Nothing |
| s08 | bacterial lawn | bacterial lawn |
| s09 | bacterial lawn | bacterial lawn |
| s10 | bacterial lawn | bacterial lawn |
| s11 | nothing | Nothing |
| s12 | nothing | Nothing |
| s13 | bacterial lawn | bacterial lawn |
| s14 | bacterial lawn | bacterial lawn |

B. Demonstration Performed on an Absorbent, Hydrophilic Culture Substrate

Evaluation of the effects of EVA and of the salts of orthophosphoric acid (more particularly, KH₂PO₄) on bacterial growth and development was carried out on an absorbent, hydrophilic culture substrate incorporating EVA and impregnated with a nutrient solution comprising, among other things, a salt of orthophosphoric acid.

1. Preparation of the Absorbent, Hydrophilic Culture Substrate

The absorbent, hydrophilic culture substrate used is in this case a piece of Airlaid® obtained from the company SCA, France (reference 95NN81) and having a declared weight of 95 g·m⁻², for a thickness of 2 mm.

EVA particles, whose surface had been coated with a pulverulent composition comprising one or more chromogenic substrate(s) (for example, 5-bromo-4-chloro-3-indolyl-alpha-glucopyranoside and/or 5-bromo-4-chloro-3-indolyl-N-methyl-α-glucopyranoside), xanthan gum and an agent that stimulates the metabolism of bacteria (MnCl₂), were immobilized within the thickness of this cellulose fiber material.

Xanthan gum is used in order to increase somewhat the absorptivity of Airlaid® and to generate a gel that will facilitate implantation and retention of bacteria within the thickness of the fibrous material. MnCl₂ makes it possible to stimulate bacterial metabolism. 5-Bromo-4-chloro-3-indolyl-α-glucopyranoside and 5-bromo-4-chloro-3-indolyl-N-methyl-α-glucopyranoside, under the hydrolytic action of an α-glucosidase, release chromophores of blue/green color that are visible to the naked eye. These chromogenic substrates thus make it possible to label the bacterial colonies expressing α-glucosidase activity which might develop on this culture substrate, for example such as colonies of S. aureus.

More precisely, the tests were carried out with square pieces of Airlaid®, with a side of 2.7 cm. These cellulose fiber pieces were dry-impregnated with a powder composition comprising (for about 100 g of composition):
80 g of EVA, with an average granulometry of the order of 60-80 μm.
20 g of xanthan gum, with an average granulometry of the order of 20-30 μm.
0.533 g of 5-bromo-4-chloro-3-indolyl-N-methyl-α-glucopyranoside and 0.3 g of 5-bromo-4-chloro-3-indolyl-α-glucopyranoside, with an average granulometry of the order of 20-30 μm,
0.027 mg of MnCl₂, with an average granulometry of the order of 20-30 μm.

To facilitate handling of this powder composition, in particular to obtain a good, homogeneous distribution of each constituent in the absorbent substrate, the latter was submitted beforehand to a hot mixing process in order to agglomerate the particles together.

With suitable heating and stirring, the surface of the EVA particles is made sticky and the particles of the other constituents of the powder composition will adhere to it. Heating is carried out at a temperature of the order of 50-60° C., which leads to softening of the EVA particles and makes their surface sticky. Mixing is carried out mechanically.

This powder composition thus assembled hot contains EVA particles, more or less well separated, whose surface is partially coated with particles of 5-bromo-4-chloro-3-indolyl-N-methyl-α-glucopyranoside and 5-bromo-4-chloro-3-indolyl-α-glucopyranoside, particles of xanthan gum and particles of MnCl₂. It is then transferred into the thickness of a sheet of Airlaid® by a technique of dry impregnation as described for example in WO 2015/044605, WO 2010/001043 and WO 99/22920. This technique consists of dusting the sheet of Airlaid® with the powder composition and causing the particles of said composition to vibrate under the action of an alternating electric field; the particles penetrate and then gradually sink into the cavities of the fibrous body.

Once impregnated, the sheet of Airlaid® is calendered between two sheets of parchment paper, the impregnated face being turned away from the heating plate. This calendering is carried out at a pressure of 4 bar and at 80° C., for 1-2 minutes.

The sheet of Airlaid® is cut into pieces with sides of 2.7 cm. Each of these squares of Airlaid® contains about 0.06 g of powder, containing about 80% of EVA.

2. Bacterial Strains Used, and Preparation of the Nutrient Solution

Strains of Staphylococcus aureus were used, in this case a methicillin-sensitive strain (MSSA) obtained from the applicant's collection. In the context of this test, the nutrients necessary for the growth and development of these bacteria are supplied by human serum (obtained from EFS) diluted with PBS. This nutrient solution aims to reproduce the nutritional qualities of the wound exudates.

Various synthetic or reconstituted exudate compositions are described in the scientific literature and may be used for reproducing the test carried out by the inventors. A culture broth with poor nutritional qualities or else peptonized water may also be used for this same purpose.

For evaluating the effect of the concentration of KH₂PO₄ on the growth and development of bacteria subjected to the presence of EVA (in this case contained within the thickness of the piece of Airlaid), serum diluted with PBS is supplemented with KH₂PO₄: 0 g·L⁻¹, 3 g·L⁻¹ 4 g·L⁻¹ 5 g·L⁻¹ and 6 g·L⁻¹ (as final concentrations).

3. Execution of the Test, and Results Obtained

100 μL of a cellular preparation concentrated to 10⁶ CFU.mL⁻¹ (in tryptone salt) is added to 900 μL of serum diluted with PBS, with or without KH₂PO₄. The pieces of Airlaid® are soaked in this solution and then incubated in a jar for 17 hours at 32° C., with a little water in the jar to prevent drying-out.

After incubation, the pieces of culture substrate were examined and the following findings were made:
The pieces of Airlaid® seeded with bacteria and a serum diluted with PBS, lacking KH₂PO₄, are colorless. No colony grew there.
A green-colored hue is observed on the pieces seeded with bacteria and a serum diluted with PBS supplemented with KH₂PO₄. A particularly pronounced coloration is found for concentrations of KH₂PO₄ of 3 g·L⁻¹ and 5 g·L⁻¹. This coloration is even more intense at 4 g·L⁻¹ of KH₂PO₄. At 6 g·L⁻¹ of KH₂PO₄, it is barely observable.

C. Demonstration in Liquid Culture Medium

Evaluation of the effects of EVA and of the salts of orthophosphoric acid on bacterial growth and development was also carried out in liquid medium. The aim is more particularly to evaluate the effect of EVA and KH₂PO₄ on the kinetics of bacterial growth.

For this purpose, 1 mL of a suspension of Staphylococcus aureus (300 CFU.mL⁻¹) obtained from the applicant's collection was cultured at 37° C. in 9 mL of different nutrient solutions:
serum diluted with PBS,
serum diluted with PBS, and comprising 18.5 g·L⁻¹ of EVA,
serum diluted with PBS, and comprising 18.5 g·L⁻¹ of EVA and 4 g·L⁻¹ of KH₂PO₄.

After each hour elapsed, a cell count is performed for a culture tube of each series. For this purpose, the cells of each tube are transferred onto an agar dish chromID® S. aureus (bioMérieux, France). The dishes are incubated for 24 hours at 37° C., before counting the colonies formed.

The three growth kinetics thus measured are presented in the form of curves in FIG. 1.

These results confirm those obtained previously, on a solid culture medium. EVA has a bacteriostatic effect. This bacteriostatic effect may be inhibited by salts of orthophosphoric acid, such as KH₂PO₄. Using KH₂PO₄ in an amount by weight corresponding to the order of ⅕ of the amount of EVA can effectively counteract the bacteriostatic effect of EVA.

### Example 2: Preparation of Biosensors According to the Invention and Evaluation of their Capacity for Detecting and Identifying Bacteria

A. Preparation and Assembly

The following example illustrates the production of a biosensor according to the invention, according to a particular embodiment.

In this particular embodiment, said biosensor is prepared from a piece made of hydrophilic absorbent fibrous material. Within the thickness of this material and fixed to its fibers, EVA particles retain and expose on their surface a pulverulent mixture combining:
a salt of orthophosphoric acid (KH₂PO₄),
two chromogenic substrates of α-glucosidase (5-bromo-4-chloro-3-indolyl-N-methyl-α-glucopyranoside and 5-bromo-4-chloro-3-indolyl-α-glucopyranoside)
a gelling agent (xanthan gum), and
an activator of bacterial metabolism (MnCl₂).

The EVA particles coated with said pulverulent mixture are prepared beforehand by hot mixing of a pulverulent composition of dissociated particles, comprising (for about 100 g of composition):
80 g of EVA,
20 g of xanthan gum,
6.67 g of KH₂PO₄,
0.533 g of 5-bromo-4-chloro-3-indolyl-N-methyl-α-glucopyranoside and 0.3 g of 5-bromo-4-chloro-3-indolyl-α-D-glucopyranoside, and
0.027 g of MnCl₂.

This pulverulent mixture is submitted to a hot mixing process in order to obtain a composition of agglomerated powders, used in the same conditions as for the hot mixing described above.

FIG. 2 shows two photographs taken by scanning electron microscopy of the pulverulent composition of dissociated particles, before hot mixing.

FIG. 3 shows two photographs taken by scanning electron microscopy of a composition of agglomerated powders according to the invention, clearly showing the EVA particles, on the surface of which finer particles are distributed, in this case particles of KH₂PO₄, 5-bromo-4-chloro-3-indolyl-N-methyl-α-glucopyranoside and 5-bromo-4-chloro-3-indolyl-N-methyl-α-glucopyranoside, xanthan gum and MnCl₂.

The composition of agglomerated powders thus prepared is then transferred into the thickness of a sheet of Airlaid® by a technique of dry impregnation, as detailed above.

Once impregnated, the sheet of Airlaid® is calendered between two sheets of parchment paper, the impregnated face being turned away from the heating plate. This calendering is carried out at a pressure of 4 bar and at 80° C., for about 1 minute and 30 seconds.

Biosensors according to the invention are cut from this sheet of Airlaid® dry-impregnated with the composition of agglomerated powders, and then calendered. They allow detection of bacteria expressing α-glucosidase activity, as is the case notably for the Staphylococcus aureus bacteria.

Other biosensors very similar in design to the first example were also assembled. In these biosensors according to the invention, the EVA particles also expose cefoxitin on their surface, which is an antibiotic to which the methicillin-sensitive strains of Staphylococcus aureus (MSSA) are particularly sensitive, in contrast to the methicillin-resistant strains (MRSA). This second example of biosensors according to the invention allows detection and identification of strains of MRSA.

The cefoxitin is fixed on the surface of the EVA particles concomitantly with KH₂PO₄, 5-bromo-4-chloro-3-indolyl-N-methyl-α-glucopyranoside and/or 5-bromo-4-chloro-3-indolyl-α-glucopyranoside, xanthan gum and MnCl₂, by a hot mixing process. For this purpose, the aforementioned pulverulent composition of dissociated particles also comprises 6.6 mg of cefoxitin.

B. Evaluation of the Above Biosensors for Detection and Identification of Staphylococcus aureus

Biosensors as described above and incorporating a composition of agglomerated powders according to the invention, comprising EVA particles on the surface of which the following are distributed:
KH₂PO₄,
5-bromo-4-chloro-3-indolyl-N-methyl-α-glucopyranoside,
5-bromo-4-chloro-3-indolyl-α-glucopyranoside,
xanthan gum and
MnCl₂,
   were tested for their ability to detect strains of Staphylococcus aureus, in this case methicillin-sensitive strains (MSSA) obtained from the applicant's collection.

For this purpose, 1 mL of serum diluted with PBS, comprising 10⁵ CFU of a strain of MSSA (or 1 mL of serum diluted with PBS free from bacteria, for the control biosensors) was deposited on squares of biosensors, with side of 2.7 cm.

The pieces of Airlaid® are then incubated in a jar for 17 hours at 32° C., with a little water in the jar to prevent drying-out.

After incubation, the biosensors were examined and the following findings were made:
The control biosensors seeded only with serum diluted with PBS remain colorless.
A green-colored hue is observed on the biosensors without cefoxitin, seeded with MSSA bacteria and the serum diluted with PBS.
The biosensors containing cefoxitin and seeded with MSSA bacteria and the serum diluted with PBS remain colorless.

1. A biosensor comprising a piece of absorbent hydrophilic material on which, on the surface and/or within the thickness thereof, a composition of agglomerated powders is immobilized, said composition comprising ethylene-vinyl acetate (EVA) particles having a surface partially coated at least with a salt of orthophosphoric acid and a visual indicator of microbiological growth.

2. The biosensor as claimed in claim 1, in which the salt of orthophosphoric acid is selected from potassium dihydrogen phosphate (KH₂PO₄) and sodium dihydrogen phosphate (NaH₂PO₄).

3. The biosensor as claimed in claim 1, in which EVA and KH₂PO₄ are present in a KH₂PO₄/EVA weight ratio between 1:25 and 1:8.

4. The biosensor as claimed in claim 1, in which EVA and NaH₂PO₄ are present in an NaH₂PO₄/EVA weight ratio between 1:15 and 1:3.

5. The biosensor as claimed in claim 1, in which the EVA has a vinyl acetate content by weight from 10 to 40%.

6. The biosensor as claimed in claim 1, in which the surface of the EVA particles is also coated with a gelling agent.

7. The biosensor as claimed in claim 6, in which the gelling agent is selected from agar, agarose, guar gum and xanthan gum.

8. The biosensor as claimed in claim 1, in which the surface of the EVA particles is also coated with MnCl₂.

9. The biosensor as claimed in claim 1, in which the surface of the EVA particles is also coated with a selective agent for antibiotic and/or antifungal effects.

10. The biosensor as claimed in claim 1, in which the piece of absorbent hydrophilic material is a piece of nonwoven.

11. A composition of agglomerated powders comprising EVA particles having a surface partially coated at least with a salt of orthophosphoric acid and a visual indicator of microbiological growth.

12. The composition as claimed in claim 11, in which the salt of orthophosphoric acid is selected from potassium dihydrogen phosphate (KH₂PO₄) and sodium dihydrogen phosphate (NaH₂PO₄).

13. The composition as claimed in claim 11, in which EVA and KH₂PO₄ are present in a KH₂PO₄/EVA weight ratio between 1:25 and 1:8.

14. The composition as claimed in claim 11, in which EVA and NaH₂PO₄ are present in an NaH₂PO₄/EVA weight ratio between 1:15 and 1:3.

15. The composition as claimed in claim 11, in which the EVA has a vinyl acetate content by weight from 10 to 40%.
